(19) United States
(12) Patent Application Publication
(10) Pub. No.: US 2007/0292481 A1
(43) Pub. Date: Dec. 20, 2007

(54) Packaging of Food Products with Pullulan Films

(76) Inventors: Andrew J. Hoffman, Mount Zion, IL (US); Michael D. Harrison, Decatur, IL (US); Shiji Shen, Forsyth, IL (US); Penelope A. Patton, Decatur, IL (US)

Correspondence Address: WILLIAMS, MORGAN & AMERSON, 10333 RICHMOND, SUITE 1100, HOUSTON, TX, 77042, US

(21) Appl. No.: 11/424,586

(22) Filed: Jun. 16, 2006

(51) Int. Cl. A61K 47/00; A23G 3/00

(52) U.S. Cl. 424/439; 426/660

(57) ABSTRACT

An edible article comprises a food product and a film that encloses the food product. The film comprises a major amount of pullulan on a dry solids basis, and a minor amount of at least two of glycerol, propylene glycol, and sorbitol. The edible article can be manufactured by preparing a film-forming composition that comprises a major amount of pullulan on a dry solids basis, and a minor amount of at least two of glycerol, propylene glycol, and sorbitol; forming the film-forming composition into a film; and enclosing a food product with the film.

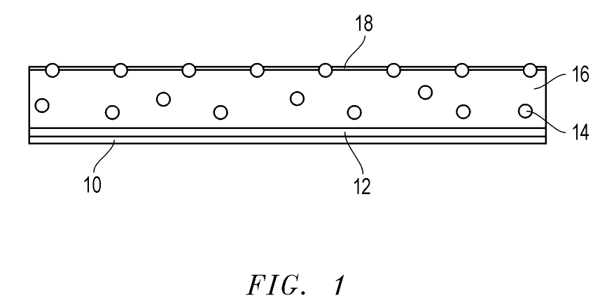

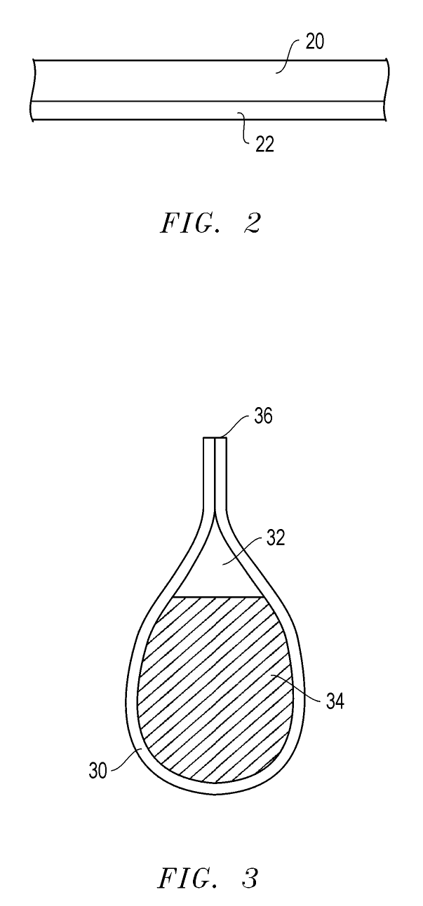

### BACKGROUND OF THE INVENTION

Some types of packaging material can be dissolved in water. For example, water soluble pouches made from polyvinyl alcohol (PVOH) film have been used to package pre-weighed farm chemicals and concrete additives. These PVOH pouches can be added to tanks or mixers, where the packaging material dissolves and the contents are released. PVOH pouches have also been used with pre-weighed laundry soap and dishwashing detergent. However, PVOH is not a food ingredient, so the PVOH technology has thus far been limited to non-food applications.

Edible films have been made from other film-forming polymers such as pullulan. For example, edible strips containing pullulan and a breath-freshening agent have been sold for human consumption. Cough medicines, vitamins, and dietary supplements have also been supplied in the form of edible strips.

Pullulan has a number of properties that make it suitable for use in edible compositions. However, one problem with pullulan films is their limited ability to elongate without breaking. This problem limits the ability of pullulan films to envelop other materials, as opposed to having other materials interspersed in the film itself. A survey of tensile strength and elongation properties of packaging films indicates that strength above 1,000 gram force and elongation of greater than 50% is likely to give pullulan-based films suitable for commercial packaging.

There is a need for improved methods of enclosing or packaging other materials in pullulan-based films or compositions.

### SUMMARY OF THE INVENTION

One aspect of the invention is an edible article that comprises a food product and a water-soluble film that encloses the food product. The film consists essentially of a major amount of pullulan on a dry solids basis, and a minor amount of more than one member selected from the group consisting of glycerol, propylene glycol, and sorbitol. “Consists essentially of” in this context means that the composition is essentially free of polysaccharides other than those listed.

In some embodiments of the invention, the film comprises about 35-80% by weight pullulan on a dry solids basis. In some embodiments, the film comprises a plasticizer mixture included at up to about 40% by weight. The plasticizer mixture in some embodiments uses a combination of glycerol, propylene glycol, and sorbitol. The film optionally can further comprise citric acid, starch or a starch derivative (such as dextrin or maltodextrin), alginate, xanthan gum, modified cellulose, polydextrose, or a combination of two or more thereof.

Another aspect of the invention is a method for making an edible article. The method comprises preparing a film-forming composition that consists essentially of a major amount of pullulan on a dry solids basis, and a minor amount of more than one member selected from the group consisting of glycerol, propylene glycol, and sorbitol; forming the film-forming composition into a water-soluble film; and enclosing a food product with the film. The components of the film-forming composition can be as described above.

In some embodiments of the invention, the film can be stretched longitudinally by at least about 50%, or at least about 100%, without breaking. In one embodiment, the food product can be enclosed by placing the food product between two pieces of film and heat-sealing the two pieces of film to form a sealed enclosure around the food product. Alternatively, the food product can be enclosed by placing the food product between two pieces of film and applying moisture and pressure to at least portions of the film to form a sealed enclosure around the food product. One specific method of enclosing that can be used is vacuum-forming the film around the food product.

### DESCRIPTION OF SPECIFIC EMBODIMENTS

The present invention relates to edible articles which contain a food product and can be consumed orally or dissolved (entirely or partially) in water. These articles have an outer layer or surface made from a film-forming composition, and the food product is enclosed inside the outer layer.

The film-forming composition comprises a major amount of pullulan on a dry solids basis. (“A major amount” in this context means that the composition contains more pullulan on a dry solids basis than any other component.) In one embodiment of the invention, the film-forming composition comprises about 35-80% by weight pullulan on a dry solids basis. Optionally, in some embodiments of the invention, other film-forming materials can be included in the film-forming composition as well, such as alginates, xanthan gum, modified cellulose, polydextrose, starch or a starch derivative (such as dextrin or maltodextrin), and combinations of two or more such materials. Inclusion of one or more of these polymers can enhance film strength and reduce cost as compared to pullulan-only compositions.

The film-forming composition also includes a minor amount of plasticizer, in particular at least two of the plasticizers glycerol, propylene glycol, and sorbitol. (“A minor amount” in this context means that the composition contains less total plasticizer than it does pullulan on a dry solids basis.) In one embodiment of the invention, the film-forming composition comprises all three of the plasticizers: glycerol, propylene glycol, and sorbitol. Each of these materials is commercially available. Optionally, in some embodiments, the composition can also include other plasticizers. In one embodiment of the invention, the film-forming composition comprises a plasticizer mixture at up to about 40% by weight.

A 20% d.s. pullulan solution in water that does not contain any plasticizer, after being cast on Mylar and then dried to residual moisture of 10% or less, results in a clear film that can be peeled away from the Mylar. The film exhibits high tensile strength, but can only be stretched and elongated about 10% in length before it breaks.

In general, pullulan-containing films that also contain plasticizers exhibit increased strength and elongation compared to pullulan films that do not contain plasticizers, up to a point. However, increasing the plasticizer content of a pullulan film beyond this level often leads to greatly decreased tensile strength. For example, addition of individual food grade plasticizers to a pullulan polymer solution prior to casting and drying gave films with elongations above 10%, but at the expense of greatly reduced tensile strength.

Surprisingly, it has been found that pullulan compositions that include at least two of the plasticizers glycerol, propylene glycol and sorbitol can be used to produce pullulan films that have high elongation and high tensile strength, even at relatively high plasticizer concentrations. In at least some embodiments of the invention, the film can be elongated at least about 50%, and in some cases at least about 100%, without breaking. In certain embodiments, the elongation without breaking is at least about 200%, or at least about 300%. In some embodiments of the invention, these enhancements to the elongation properties of the film are achieved without a substantial reduction in tensile strength.

The composition optionally can also contain one or more additives that are suitable for use in foods, such as fillers, surfactants, stabilizers, organic acids (such as citric acid), and flavorings.

One specific embodiment of the invention is a water-soluble, edible film-forming composition that consists essentially of a major amount of pullulan on a dry solids basis, and a minor amount of more than one member selected from glycerol, propylene glycol, and sorbitol. This composition can be formed into films having a thickness of less than 2.2 mils (0.0022 inches or 0.056 mm) that will exhibit tensile strength in excess of 1,000 grams force and elongation to break in excess of 50%.

In another specific embodiment of the invention, the water-soluble, edible film-forming composition consists essentially of a major amount of pullulan on a dry solids basis and minor amounts of (i) a co-polysaccharide selected from the group consisting of alginates, cellulose ethers, modified starches, and combinations thereof, and (ii) more than one member selected from the group consisting of glycerol, propylene glycol, and sorbitol. (“A minor amount” in this context means that the composition contains less total plasticizer than it does pullulan on a dry solids basis, and also contains less total co-polysaccharide than it does pullulan on a dry solids basis.) The composition can be formed into a film having a thickness of less than 2.2 mils that will exhibit tensile strength in excess of 1,000 grams force and elongation to break in excess of 50%.

Techniques of forming films using pullulan compositions are well known in the art. For example, an aqueous pullulan solution can be cast onto a flat surface, and then heated and dried to form the film. Methods for controlling the thickness of the film are also well known.

There are many different ways that the film-forming composition can be used to enclose a food product. For example, a film can be formed into a pouch, the food product can be placed in the pouch, and then the opening in the pouch can be sealed, for example by application of heat and/or moisture. One specific technique that can be used is vacuum-forming the film around the food product. Vacuum forming has the advantage of requiring less extreme folding and bending of the film web under tension, as compared to some other methods of enclosing a product with a film.

The food grade films of the present invention can have the tensile strength and elongation properties necessary to successfully produce edible packages on commercial vacuum-forming equipment. They also can have the ability to form many different shapes and work on complex molds more successfully than at least some other commercial film-forming materials. In some embodiments, the films exhibit tensile strength in excess of 1,000 grams force and elongation to break in excess of 50%.

A wide variety of food products can be encapsulated, including ones that need to be dissolved or dispersed in water for cooking and ones that are supplied in single-serve packages for human consumption. Examples of such food products include, but are not limited to powdered beverage mixes (such as cocoa drink products, soft drink products, and cider drink products), powdered cheese products, powdered egg products, candy, dry soup and casserole mixes, food dyes and spices. The food product itself can be, but does not necessarily have to be, water-soluble.

### EXAMPLES

The following experimental methods were used in the examples described below.

### Preparation of Pullulan and other Polymer Solutions

Polymer solutions were prepared to have less than 10,000 centipoises viscosity. Water was placed in a vessel and agitated, and then the dry polymer powder was added to the vortex of the stirring liquid over time. Stirring at 100-1000 rpm was continued for 30-60 minutes, then the solution was allowed to rest for at least two hours prior to use.

### Incorporation of Plasticizers and other Additives

Polymer solutions were blended as needed to give the desired ratios and concentrations, and then the oligomers, plasticizers, and other additives were added neat to the polymer solutions with mixing over time.

### Film Casting and Drying

Aqueous solutions were cast onto Mylar film by machine or by hand using drawdown bars with a gap of either 20 or 40 mils at a rate of about 1 meter per second. The Mylar film was taped onto 0.50 in thick glass sheets prior to solution casting. The whole assembly (casting, Mylar, and glass) was placed into a controlled drying chamber set for 140° F. and 30% relative humidity (RH) for 2-3 hours to dry the pullulan films.

### Film Conditioning and Testing

Films were conditioned in a controlled environment room set for 70° F. and 50% RH for 1 to 5 days (average 3) prior to testing. Samples were transferred to the testing area in Zip-Loc® bags. Samples were tested and evaluated for tensile strength (gram force) and % elongation using a small laboratory Instron physical testing unit. In the test, a metal probe with an elliptical tip is forced thru the plane of a tightly held piece of film. The amount of force required to break the film, and the distance the probe travels to break the film are used to calculate the material properties.

### Pouch Production via Vacuum Forming

A die was selected and placed on the table under a vertically-movable frame. A sheet of film (7 in×11 in) was placed on the bottom part of the frame. The upper part of the frame was lowered and locked onto the bottom part. A vacuum was pulled through the die, the film was lowered onto the die and sucked into it, forming a pouch, and then the pouch was filled with selected material.

### Heat Sealing

A second piece of film was laid smoothly on top of the pouch. A hot iron (200-300° F.) was manually pressed onto both pieces of film at the edge of the filled area. The iron was held in place for 2-5 seconds.

### Moisture Sealing

A second piece of film was wrapped around a block and was quickly pressed into a damp paper towel. The lightly moisturized film was pressed onto the previously formed pouch for about 2-5 seconds.

### Example 1

Commercially available pullulan from Hayashibara (PI-20) was used to prepare films with one or more of the following additives: glycerol, propylene glycol (PPG), Sorbitol Special (SorbS; SPI Pharma; 40-55% sorbitol, 15-30% sorbitol anhydrides, and 1-10% mannitol), Nu-Col 2004 (NC2004; Tate & Lyle modified starch), Star-Dri 5 (Tate & Lyle maltodextrin), MiraSperse 2000 (MS2000; Tate & Lyle modified starch), DuraGel (Tate & Lyle modified starch), TenderJel C (Tate & Lyle modified starch), and sodium alginate. The specific compositions are shown in Table 1A.

TABLE 1A

| Ref. | total | | | | % | % | |
|---|---|---|---|---|---|---|---|
| No. | % d.s. | % pullulan | additive 1 | % additive 1 | glycerol | PPG | % SorbS |
| 1-1 | 20.0% | 80.0% | | | 20.0% | | |
| 1-2 | 20.0% | 48.0% | Na alginate | 12.0% | 10.0% | 10.0% | 20.0% |
| 1-3 | 20.0% | 48.0% | NC2004 | 12.0% | 10.0% | 10.0% | 20.0% |
| 1-4 | 20.0% | 48.0% | Star-Dri 5 | 12.0% | 10.0% | 30.0% | |
| 1-5 | 20.0% | 80.0% | Star-Dri 5 | 20.0% | | | |
| 1-6 | 20.0% | 48.0% | Na alginate | 12.0% | 10.0% | 30.0% | |
| 1-7 | 20.0% | 80.0% | TenderJel C | 20.0% | | | |
| 1-8 | 20.0% | 48.0% | DuraGel | 12.0% | 10.0% | 30.0% | |
| 1-9 | 20.0% | 80.0% | DuraGel | 20.0% | | | |
| 1-10 | 34.0% | 48.9% | MS2000 | 12.0% | 9.8% | 29.3% | |
| 1-11 | 37.0% | 41.9% | MS2000 | 24.6% | 8.3% | 25.1% | |
| 1-12 | 20.0% | 48.0% | NC2004 | 12.0% | 10.0% | 30.0% | |
| 1-13 | 20.0% | 80.0% | MS2000 | 20.0% | | | |
| 1-14 | 20.0% | 80.0% | NC2004 | 20.0% | | | |
| 1-15 | 20.0% | 80.0% | | | | 20.0% | |
| 1-16 | 20.0% | 80.0% | | | 20.0% | | |

The results of tests of the film properties are given in Table 1B.

TABLE 1B

| | Film | | | | |
|---|---|---|---|---|---|
| Ref | Thickness | Force | Elongation | Force | |
| No. | (mil) | (gram) | (percent) | (coeff. var.) | N |
| 1-1 | 2.0 | 2,018 | 15% | 15% | 3 |
| 1-2 | 2.2 | 1,068 | 62% | 9% | 5 |
| 1-3 | 2.2 | 841 | 57% | 15% | 5 |
| 1-4 | 2.6 | 1,666 | 22% | 5% | 4 |
| 1-5 | 2.0 | 3,174 | 12% | 16% | 4 |
| 1-6 | 1.7 | 1,069 | 7% | 32% | 5 |
| 1-7 | 2.4 | 2,114 | 5% | 12% | 4 |
| 1-8 | 1.6 | 827 | 5% | 10% | 4 |
| 1-9 | 3.0 | 1,818 | 4% | 3% | 4 |
| 1-10 | 2.8 | 763 | 4% | 3% | 5 |
| 1-11 | 3.1 | 755 | 4% | 4% | 5 |
| 1-12 | 1.8 | 569 | 3% | 22% | 4 |
| 1-13 | 2.0 | 1,113 | 2% | 12% | 4 |
| 1-14 | 2.0 | 981 | 2% | 34% | 4 |
| 1-15 | 2.4 | 857 | 2% | 15% | 5 |
| 1-16 | 2.4 | 1,988 | 17% | 4% | 4 |

Films containing a combination of three plasticizers (glycerol, propylene glycol and sorbitol special) gave elongations above 50% at high strength in samples 1-2 and 1-3.

### Example 2

Films were prepared containing pullulan and additional ingredients shown in Table 2A.

TABLE 2A

| Ref. | total % | % | % Na | % | % | % | | % | % citric |
|---|---|---|---|---|---|---|---|---|---|
| No. | d.s. | pullulan | alginate | Star-Dri 5 | NC2004 | glycerol | % PPG | SorbS | acid |
| 2-1 | 20.0% | 80.0% | | | | 20.0% | | | |
| 2-2 | 22.8% | 56.0% | 2.8% | 11.2% | | 5.0% | 10.0% | 15.0% | |
| 2-3 | 25.0% | 56.0% | 2.8% | 11.2% | | | 12.0% | 18.0% | |
| 2-4 | 25.0% | 56.0% | 2.8% | 11.2% | | | 10.0% | 20.0% | |
| 2-5 | 30.0% | 56.0% | 2.8% | 8.4% | 2.8% | 0.0% | 10.0% | 20.0% | 0.0% |
| 2-6 | 30.0% | 56.0% | 2.8% | 8.4% | 2.8% | | | 20.0% | 10.0% |
| 2-7 | 25.0% | 56.0% | 2.8% | 11.2% | | | | 20.0% | 10.0% |
| 2-8 | 25.0% | 56.0% | 2.8% | 11.2% | | 5.0% | 10.0% | 15.0% | |
| 2-9 | 25.0% | 56.0% | 2.8% | 11.2% | | | 10.0% | 20.0% | |
| 2-10 | 25.0% | 48.0% | 2.4% | 9.6% | | 10.0% | 10.0% | 20.0% | |
| 2-11 | 30.0% | 56.0% | 2.8% | 8.4% | 2.8% | 0.0% | 12.0% | 18.0% | |
| 2-12 | 25.0% | 53.2% | 2.8% | 11.2% | 2.8% | 5.0% | 10.0% | 15.0% | |
| 2-13 | 25.0% | 56.0% | 2.8% | 11.2% | | 5.0% | 10.0% | 15.0% | |
| 2-14 | 30.0% | 48.0% | 2.4% | 7.2% | 2.4% | 10.0% | 10.0% | 20.0% | |
| 2-15 | 30.0% | 48.0% | 2.4% | 7.2% | 2.4% | 10.0% | 10.0% | 20.0% | |
| 2-16 | 24.0% | 57.0% | 2.6% | 10.4% | | 5.0% | 10.0% | 15.0% | |
| 2-17 | 30.0% | 48.0% | 2.4% | 7.2% | 2.4% | 10.0% | 10.0% | 20.0% | |
| 2-18 | 25.0% | 50.4% | 2.4% | 7.2% | | 5.0% | 10.0% | 15.0% | 10.0% |
| 2-19 | 25.0% | 52.0% | 2.6% | 10.4% | | 5.0% | 5.0% | 15.0% | 10.0% |
| 2-20 | 25.0% | 56.0% | 2.8% | 11.2% | | 5.0% | 5.0% | 15.0% | 5.0% |
| 2-21 | 26.4% | 48.0% | 2.4% | 9.6% | | 5.0% | 10.0% | 15.0% | 10.0% |
| 2-22 | 24.0% | 57.0% | 2.6% | 10.4% | | 5.0% | 10.0% | 15.0% | |
| 2-23 | 24.0% | 48.0% | 2.4% | 9.6% | | 5.0% | 10.0% | 15.0% | 10.0% |
| 2-24 | 25.0% | 56.0% | 2.8% | 11.2% | | 5.0% | 10.0% | 15.0% | |

Tests were performed to determine the properties of these films, and the results are given in Table 2B.

TABLE 2B

| | Film | | | Force | |
|---|---|---|---|---|---|
| | Thickness | Force | Elongation | (coeff. | |
| Ref. No. | (mil) | (gram) | (percent) | var.) | N |
| 2-1 | 2.8 | 1,644 | 10% | 9% | 5 |
| 2-2 | 5.9 | 3,820 | 90% | 4% | 4 |
| 2-3 | 5.1 | 2,799 | 111% | 3% | 3 |
| 2-4 | 5.6 | 2,766 | 103% | 17% | 5 |
| 2-5 | 5.4 | 2,570 | 93% | 16% | 5 |
| 2-6 | 5.6 | 2,417 | 83% | 9% | 3 |
| 2-7 | 5.5 | 2,396 | 67% | 3% | 5 |
| 2-8 | 5.2 | 2,374 | 122% | 3% | 4 |
| 2-9 | 6.1 | 2,080 | 266% | 5% | 5 |
| 2-10 | 6.3 | 1,908 | 161% | 5% | 5 |
| 2-11 | 5.5 | 1,892 | 151% | 9% | 5 |
| 2-12 | 4.3 | 1,581 | 271% | 4% | 3 |
| 2-13 | 4.4 | 1,500 | 133% | 10% | 5 |
| 2-14 | 2.2 | 1,277 | 65% | 11% | 4 |
| 2-15 | 2.2 | 1,264 | 55% | 2% | 5 |
| 2-16 | 5.5 | 1,188 | 275% | 9% | 5 |
| 2-17 | 2.1 | 1,166 | 60% | 4% | 5 |
| 2-18 | 4.9 | 1,156 | 342% | 5% | 5 |
| 2-19 | 5.5 | 1,127 | 193% | 9% | 5 |
| 2-20 | 4.7 | 1,099 | 215% | 14% | 5 |
| 2-21 | 6.0 | 1,056 | 213% | 5% | 5 |
| 2-22 | 4.7 | 1,028 | 231% | 3% | 5 |
| 2-23 | 5.4 | 1,025 | 350% | 8% | 5 |
| 2-24 | 2.1 | 1,019 | 105% | 10% | 5 |

The films made with STAR-DRI 5 maltodextrin and sodium alginate (and optionally Nu-Col 2004) with pullulan as the predominant polymer showed high tensile strength. High elongations were seen in films containing glycerol, propylene glycol and sorbitol (and optionally citric acid) with pullulan as the predominant polymer. Variations in thickness resulted in films with tensile strength in excess of 1,000 grams force and elongation to break in excess of 50%.

### Example 3

The following films were prepared for testing on a laboratory vacuum forming packaging apparatus:

TABLE 3A

| Ref. | Total % | % | % Na | % Star- | % | | % | other | % other |
|---|---|---|---|---|---|---|---|---|---|
| No. | d.s. | pullulan | alginate | Dri 5 | glycerol | % PPG | SorbS | additive(s) | additive(s) |
| 1 | 20.0 | 80.0 | | 20.0 | | | | | |
| 2 | 24.8 | 60.0 | 3.0 | 12.0 | 6.3 | 6.3 | 12.5 | | |
| 3 | 25.0 | 56.0 | 2.8 | 11.2 | | 10.0 | 20.0 | | |
| 4 | 25.0 | 56.0 | 2.8 | 11.2 | 5.0 | 10.0 | 15.0 | | |
| 5 | 25.0 | 50.4 | 2.4 | 7.2 | 5.0 | 10.0 | 15.0 | citric acid | 10.0 |
| 6 | 24.1 | 52.0 | 2.6 | 7.8 | 10.0 | 10.0 | 15.0 | NC2004/ | 2.6/10.0 |
| | | | | | | | | citric acid | |

Tests were performed to evaluate the film properties, and the results are shown in Table 3B

TABLE 3B

| | Film | | | | |
|---|---|---|---|---|---|
| | Thickness | Force | Elongation | Force | |
| Film Ref. No. | (mil) | (gram) | (%) | (% coeff. var.) | N |
| 1 | 2.1 | 3,174 | 12 | 8 | 5 |
| 2 | 2.6 | 1,600 | 47 | 14 | 5 |
| 3 | 2.2 | 2,766 | 100 | 17 | 5 |
| 4 | 5.9 | 1,500 | 130 | 10 | 5 |
| 5 | 6.0 | 1,131 | 250 | 5 | 5 |
| 6 | 5.8 | 879 | 216 | 9 | 5 |

The following dies were used in the vacuum packaging tests:

Die 1) Half egg-shaped: 2.50 in L by 1.88 in W by 0.69 in D—maximum depth tapered down from edge.

Die 2) Rectangular: 4.50 in L by 2.75 in W by 0.50 in D—uniform depth straight down from edge.

Die 3) Half tube: 1.88 in L by 0.75 in W by 0.50 in D—maximum depth tapered down from edge.

Die 4) Seven half cylinders: 2.00 in L by 0.75 in W by 0.50 in D—maximum depth tapered down from edge, each cylinder spaced 0.38″ apart.

Die 5) Tapered Square: 1.88 in L by 1.88 in W by 0.75 in D—maximum depth tapered down from edge.

Various food products were enclosed with the films as described below, forming edible, water soluble packages.

Example 3-1—Film #6 was successfully vacuum formed using Die #1 and about 12 grams of finely powdered ALLEGGRA® FS74 egg product was filled in the pouch. Film #1 was successfully used to close the package by heat sealing.

Example 3-2—Film #5 was successfully vacuum formed using Die #1 and about 20 grams of finely powdered Swiss Miss® Hot Cocoa Mix was filled in the pouch. Film #1 was unsuccessfully used to close the package due to fracture during heat sealing.

Example 3-3—Film #5 was successfully vacuum formed using Die #1 and about 12 grams of finely powdered ALLEGGRA® FS74 egg product was filled in the pouch. Film #5 was successfully used to close the package by heat sealing.

Example 3-4—Film #3 was successfully vacuum formed using Die #1 and about 20 grams of finely powdered Swiss Miss® Hot Cocoa Mix was filled in the pouch. Film #3 was successfully used to close the package by heat sealing.

Example 3-5—Film #4 was successfully vacuum formed using Die #1 and about 12 grams of finely powdered ALLEGGRA® FS74 egg product was filled in the pouch. Film #4 was successfully used to close the package by heat sealing.

Example 3-6—Film #4 was successfully vacuum formed using Die #2 and about 28 grams of finely powdered Swiss Miss® Hot Cocoa Mix was filled in the pouch. Film #4 was successfully used to close the package by heat sealing. This package was later found to have a minute hole in the deep corner of a vacuum formed region.

Example 3-7—Film #3 was successfully vacuum formed using Die #2 and about 40 grams of finely powdered Swiss Miss® Hot Cocoa Mix was filled in the pouch. Film #3 was successfully used to close the package by heat sealing.

Example 3-8—Film #4 was successfully vacuum formed using Die #2 and about 28 grams of finely powdered Swiss Miss® Hot Cocoa Mix was filled in the pouch. Film #4 was successfully used to close the package by heat sealing. This package was a redo of Example 5-6 and showed no defects.

Example 3-9—Film #1 was unsuccessfully vacuum formed using Die #2. The film shattered to bits.

Example 3-10—Film #2 was successfully vacuum formed using Die #2 and about 24 grams of finely powdered ALLEGGRA® FS74 egg product was filled in the pouch. Film #4 was successfully used to close the package by heat sealing. This package was later found to have a leak due to a heat sealing defect.

Example 3-11—Film #5 was successfully vacuum formed using Die #3 and about 5 grams of finely powdered Crystal Light® Soft Drink Mix was filled in the pouch. Film #4 was successfully used to close the package by heat sealing.

Example 3-12—Film #5 was successfully vacuum formed using Die #4 and about 5 grams of finely powdered Crystal Light® Soft Drink Mix was filled in each of seven pouches. Film #5 was successfully used to close the package by heat sealing. This film was capable of filling multiple adjacent cavities in a single vacuum forming operation.

Example 3-13—Film #6 was successfully vacuum formed using Die #4 and about 4 grams of finely powdered Alpine® Spiced Cider Sugar Free Drink Mix was filled in each of seven pouches. Film #6 was successfully used to close the package by heat sealing. This film was capable of filling multiple adjacent cavities in a single vacuum forming operation.

Example 3-14—Film #4 was successfully vacuum formed using Die #4 and about 5 grams of finely powdered Easy Mac® Cheese Powder was filled in each of seven pouches. Film #4 was successfully used to close the package by heat sealing. This film survived but seemed to be at the limit of its elongation and gave audible signs of stress during vacuum forming.

Example 3-15—Film #3 was successfully vacuum formed using Die #1 and about 17 grams of finely powdered Easy Mac® Cheese Powder was filled in the pouch. Film #3 was successfully used to close the package by heat sealing.

Example 3-16—Film #2 was successfully vacuum formed using Die #1 and about 17 grams of finely powdered Easy Mac® Cheese Powder was filled in the pouch. Film #3 was successfully used to close the package by heat sealing. This package was later found to have a leak due to a heat sealing defect.

Example 3-17—Film #4 was successfully vacuum formed using Die #2 and about 40 grams of finely powdered Easy Mac® Cheese Powder was filled in the pouch. Film #4 was successfully used to close the package by heat sealing. This package was later found to have a leak due to a heat sealing defect.

Example 3-18—Film #3 was successfully vacuum formed using Die #1 and about 17 grams of finely powdered Easy Mac® Cheese Powder was filled in the pouch. Film #3 was successfully used to close the package by water sealing.

Example 3-19—Film #5 was successfully vacuum formed using Die #5 and about 8 grams of finely powdered Easy Mac® Cheese Powder was filled in the pouch. Film #5 was successfully used to close the package by water sealing.

Example 3-20—Film #3 (at 6 mil) was successfully vacuum formed using Die #5 and about 8 grams of finely powdered Easy Mac® Cheese Powder was filled in the pouch. Film #3 (at 6 mil) was successfully used to close the package by water sealing.

Example 3-21—A blue colored and peppermint flavored film of 2 mil thickness was made using the following ingredients (all in % w/w, d.s. basis): pullulan (PI-20) 50%, tapioca dextrin (F4-800) 13%, glycerol 6%, propylene glycol 13%, and sorbitol 19%. The film was formed into a small ½ inch square pouch using a laboratory impulse sealer. Each pouch was filled with about 0.25 g of strawberry flavored Pop Rocks® candy and sealed. Thus, this test produced an edible, two-part confectionary where the immediate flavor of the film is supplanted by the flavor and sensory attributes of the Pop Rocks® candy once the film is dissolved in the mouth.

The preceding description of certain embodiments of the invention is not intended to be an exhaustive list of all possible embodiments. Persons skilled in this field will appreciate that modifications could be made to the specific embodiments described herein which would be within the scope of the following claims.

1. An edible article, comprising a food product and a water-soluble film that encloses the food product, wherein the film consists essentially of a major amount of pullulan on a dry solids basis, and a minor amount of more than one member selected from glycerol, propylene glycol, and sorbitol.

2. The edible article of claim 1, wherein the film comprises about 35-80% by weight pullulan on a dry solids basis.

3. The edible article of claim 1, wherein the film comprises glycerol, propylene glycol, and sorbitol.

4. The edible article of claim 3, wherein the film comprises about 2-30% glycerol, about 2-30% propylene glycol, and about 2-30% by weight sorbitol on a dry solids basis.

5. The edible article of claim 1, wherein the film further comprises citric acid.

6. The edible article of claim 1, wherein the film further comprises starch or a starch derivative.

7. The edible article of claim 6, wherein the starch derivative is dextrin or maltodextrin.

8. The edible article of claim 1, wherein the film further comprises alginate, xanthan gum, collagen, polydextrose, or a combination of two or more thereof.

9. The edible article of claim 1, wherein the food product is selected from powdered beverage mix, candy, powdered cheese product, powdered egg product, dry soup and casserole mixes, food dyes and spices.

10. A method for making an edible article, comprising:
preparing a film-forming composition that consists essentially of a major amount of pullulan on a dry solids basis, and a minor amount of more than one member selected from glycerol, propylene glycol, and sorbitol;
forming the film-forming composition into a water-soluble film; and
enclosing a food product with the film.

11. The method of claim 10, wherein the film can be stretched longitudinally by at least about 50% without breaking.

12. The method of claim 10, wherein the film can be stretched longitudinally by at least about 100% without breaking.

13. The method of claim 10, wherein the film-forming composition is formed into a film by casting.

14. The method of claim 10, wherein the food product is enclosed by placing the food product between two pieces of film and heat-sealing the two pieces of film to form a sealed enclosure around the food product.

15. The method of claim 10, wherein the food product is enclosed by placing the food product between two pieces of film and applying moisture and pressure to at least portions of the film to form a sealed enclosure around the food product.

16. The method of claim 10, wherein the food product is enclosed by vacuum-forming the film around the food product.

17. The method of claim 10, wherein the film-forming composition comprises about 35-80% by weight pullulan on a dry solids basis.

18. The method of claim 10, wherein the film comprises glycerol, propylene glycol, and sorbitol.

19. The method of claim 18, wherein the film-forming composition comprises about 2-30% glycerol, about 2-30% propylene glycol, and about 2-30% by weight sorbitol on a dry solids basis.

20. The method of claim 10, wherein the film-forming composition further comprises citric acid.

21. The method of claim 10, wherein the film-forming composition further comprises starch or a starch derivative.

22. The method of claim 21, wherein the starch derivative is dextrin or maltodextrin.

23. The method of claim 10, wherein the film-forming composition further comprises alginate, xanthan gum, collagen, polydextrose, or a combination of two or more thereof.

24. The method of claim 10, wherein the food product is selected from powdered beverage mix, candy, powdered cheese product, powdered egg product, dry soup and casserole mixes, food dyes and spices.

25. The method of claim 10, wherein the film exhibits tensile strength in excess of 1,000 grams force and elongation to break in excess of 50%.

26. The method of claim 10, wherein the film-forming composition further comprises a minor amount of a co-polysaccharide selected from the group consisting of alginates, cellulose ethers, modified starches, and combinations thereof, and wherein the film exhibits tensile strength in excess of 1,000 grams force and elongation to break in excess of 50%.

27. A water-soluble, edible film-forming composition consisting essentially of a major amount of pullulan on a dry solids basis, and a minor amount of more than one member selected from glycerol, propylene glycol, and sorbitol, said composition being formable into films having a thickness of less than 2.2 mils which exhibit tensile strength in excess of 1,000 grams force and elongation to break in excess of 50%.

28. A water-soluble, edible film consisting essentially of a major amount of pullulan on a dry solids basis, and a minor amount of more than one member selected from glycerol, propylene glycol, and sorbitol, and having a thickness of less than 2.2 mils wherein said film exhibits tensile strength in excess of 1,000 grams force and elongation to break in excess of 50%.

29. A water-soluble, edible film-forming composition consisting essentially of a major amount of pullulan on a dry solids basis and minor amounts of (i) a co-polysaccharide selected from the group consisting of alginates, cellulose ethers, modified starches, and combinations thereof, and (ii) more than one member selected from the group consisting of glycerol, propylene glycol, and sorbitol, said composition being formable into a film having a thickness of less than 2.2 mils which exhibits tensile strength in excess of 1,000 grams force and elongation to break in excess of 50%.

30. A water-soluble, edible film consisting essentially of a major amount of pullulan on a dry solids basis, and minor amounts of (i) a co-polysaccharide selected from the group consisting of alginates, cellulose ethers, modified starches, and combinations thereof, and (ii) more than one member selected from the group consisting of glycerol, propylene glycol, and sorbitol, and having a thickness of less than 2.2 mils wherein said film exhibits tensile strength in excess of 1,000 grams force and elongation to break in excess of 50%.

31. An edible article comprising a food product and a water-soluble film that encloses the food product, wherein the film consists essentially of a major amount of pullulan on a dry solids basis, and a minor amount of more than one member selected from glycerol, propylene glycol, and sorbitol, and wherein the film has a thickness of less than 2.2 mils and exhibits tensile strength in excess of 1,000 grams force and elongation to break in excess of 50%.

32. The edible article of claim 31, wherein the film further comprises a minor amount of a co-polysaccharide selected from the group consisting of alginates, cellulose ethers, modified starches, and combinations thereof.
